(19) United States
(12) Patent Application Publication
(10) Pub. No.: US 2022/0108773 A1
(43) Pub. Date: Apr. 7, 2022

(54) SYSTEMS AND METHODS FOR GENOME ANALYSIS AND VISUALIZATION

(71) Applicant: Baidu USA, LLC, Sunnyvale, CA (US)

(72) Inventors: Boxiang LIU, Sunnyvale, CA (US); Kaibo LIU, Mountain View, CA (US); He ZHANG, Santa Clara, CA (US); Liang ZHANG, Fremont, CA (US); Yuchen BIAN, Santa Clara, CA (US); Liang HUANG, Mountain View, CA (US)

(73) Assignee: Baidu USA LLC, Sunnyvale, CA (US)

(21) Appl. No.: 17/065,233

(22) Filed: Oct. 7, 2020

(51) Int. Cl. G16B 45/00; G16B 30/10

(52) U.S. Cl. CPC G16B 45/00; G16B 30/10

(57) ABSTRACT

COVID-19 has become a global pandemic after its inception in late 2019. SARS-CoV-2 genomes are sequenced and shared on public repositories at a fast pace. To keep up with these updates, datasets need to be refreshed and re-cleaned frequently. It may be difficult to analyze SARS-CoV-2 genomes for scientists with limited bioinformatics or programming knowledge. In the present disclosure, system and method embodiments for genome analysis and visualization are developed to address these challenges. A webserver may be used to enable simple and rapid analysis of genomes. Given a new sequence, the system may automatically predict gene boundaries and identify genetic variants, which are presented in an interactive genome visualizer and are downloadable for analysis. A command-line interface may be available for high throughput processing.

### COPYRIGHT NOTICE

A portion of the disclosure of this patent document contains material that is subject to copyright protection. The copyright owner has no objection to the facsimile reproduction by anyone of the patent document, as it appears in the Patent and Trademark Office patent file or records, but otherwise reserves all copyright rights whatsoever.

### BACKGROUND

### A. Technical Field

The present disclosure relates generally to systems and methods for visualization. More particularly, the present disclosure relates to systems and methods for genome analysis and visualization that can provide improved features, and uses.

### B. Background

The 2019 novel Coronavirus (SARS-CoV-2) caused an outbreak of viral pneumonia since late 2019 and has become a global pandemic. Despite efforts to contain its spread, SARS-CoV-2 has infected millions of patients and more than 800,000 deaths worldwide as of late August. To understand its evolution and genetics, scientists have sequenced SARS-CoV-2 genomes from patients across different age groups, genders, ethnicities, locations, and disease stages. These genomic sequences are being shared on public repositories at a rapid pace, with thousands of new sequences every week. To keep up with the latest developments, scientists need to frequently download and clean new datasets, which is ad hoc and time-consuming. On the other hand, scientists with limited knowledge in bioinformatics or programming may experience difficulty in analyzing SARS-CoV-2 genomes.

Accordingly, what is needed are systems and methods for genome analysis and visualization to address the challenges.

### BRIEF DESCRIPTION OF THE DRAWINGS

References will be made to embodiments of the disclosure, examples of which may be illustrated in the accompanying figures. These figures are intended to be illustrative, not limiting. Although the disclosure is generally described in the context of these embodiments, it should be understood that it is not intended to limit the scope of the disclosure to these particular embodiments. Items in the figures may not be to scale.

FIG. 1 depicts for a system for genome analysis and visualization, according to embodiments of the present disclosure.

FIG. 2 graphically depicts an input interface for sequence input, according to embodiments of the present disclosure.

FIG. 3 depicts an interactive genome visualizer, according to embodiments of the present disclosure.

FIG. 4 depicts an interactive genome visualizer showing a nucleotide symbol chain and a corresponding amino acid (AA) residue chain in a genome window, according to embodiments of the present disclosure.

FIG. 5 depicts an interactive genome visualizer showing two sequences in a genome window, according to embodiments of the present disclosure.

FIG. 6 depicts a mutation table showing positions, alleles and intersecting open reading frames (ORFs), according to embodiments of the present disclosure.

FIG. 7 depicts an ORF table showing predicted gene boundaries and supporting information, according to embodiments of the present disclosure.

FIG. 8 depicts an ORF table showing nucleotide and protein sequences for the selected ORF, according to embodiments of the present disclosure.

FIG. 9 depicts an interactive genome visualizer along with one or two tables without user interaction, according to embodiments of the present disclosure.

FIG. 10 depicts an interactive genome visualizer along with one or two tables dynamically shown based on user interaction, according to embodiments of the present disclosure.

FIG. 11 graphically depicts a variant call format (VCF) file, according to embodiments of the present disclosure.

FIG. 12 graphically depicts a data analysis pipeline, according to embodiments of the present disclosure.

FIG. 13 depicts a process of genome analysis in the data analysis pipeline, according to embodiments of the present disclosure.

FIG. 14 depicts a distribution of SARS-CoV-2 sequence lengths, according to embodiments of the present disclosure.

FIG. 15 depicts a distribution of sample mutations identified against a reference genome NC 045512.2, according to embodiments of the present disclosure.

FIG. 16 depicts a distribution of multi-allelic sites along the SARS-CoV-2 genome, according to embodiments of the present disclosure.

FIG. 17 depicts a simplified block diagram of a computing device/information handling system, according to embodiments of the present disclosure.

### DETAILED DESCRIPTION OF EMBODIMENTS

In the following description, for purposes of explanation, specific details are set forth in order to provide an understanding of the disclosure. It will be apparent, however, to one skilled in the art that the disclosure can be practiced without these details. Furthermore, one skilled in the art will recognize that embodiments of the present disclosure, described below, may be implemented in a variety of ways, such as a process, an apparatus, a system, a device, or a method on a tangible computer-readable medium.

Components, or modules, shown in diagrams are illustrative of exemplary embodiments of the disclosure and are meant to avoid obscuring the disclosure. It shall also be understood that throughout this discussion that components may be described as separate functional units, which may comprise sub-units, but those skilled in the art will recognize that various components, or portions thereof, may be divided into separate components or may be integrated together, including, for example, being in a single system or component. It should be noted that functions or operations discussed herein may be implemented as components. Components may be implemented in software, hardware, or a combination thereof.

Furthermore, connections between components or systems within the figures are not intended to be limited to direct connections. Rather, data between these components may be modified, re-formatted, or otherwise changed by intermediary components. Also, additional or fewer connections may be used. It shall also be noted that the terms “coupled,” “connected,” “communicatively coupled,” “interfacing,” “interface,” or any of their derivatives shall be understood to include direct connections, indirect connections through one or more intermediary devices, and wireless connections. It shall also be noted that any communication, such as a signal, response, reply, acknowledgement, message, query, etc., may comprise one or more exchanges of information.

Reference in the specification to “one or more embodiments,” “preferred embodiment,” “an embodiment,” “embodiments,” or the like means that a particular feature, structure, characteristic, or function described in connection with the embodiment is included in at least one embodiment of the disclosure and may be in more than one embodiment. Also, the appearances of the above-noted phrases in various places in the specification are not necessarily all referring to the same embodiment or embodiments.

The use of certain terms in various places in the specification is for illustration and should not be construed as limiting. A service, function, or resource is not limited to a single service, function, or resource; usage of these terms may refer to a grouping of related services, functions, or resources, which may be distributed or aggregated. The terms “include,” “including,” “comprise,” and “comprising” shall be understood to be open terms and any lists the follow are examples and not meant to be limited to the listed items. A “layer” may comprise one or more operations. The words “optimal,” “optimize,” “optimization,” and the like refer to an improvement of an outcome or a process and do not require that the specified outcome or process has achieved an “optimal” or peak state. The use of memory, database, information base, data store, tables, hardware, cache, and the like may be used herein to refer to system component or components into which information may be entered or otherwise recorded.

In one or more embodiments, a stop condition may include: (1) a set number of iterations have been performed; (2) an amount of processing time has been reached; (3) convergence (e.g., the difference between consecutive iterations is less than a first threshold value); (4) divergence (e.g., the performance deteriorates); and (5) an acceptable outcome has been reached.

One skilled in the art shall recognize that: (1) certain steps may optionally be performed; (2) steps may not be limited to the specific order set forth herein; (3) certain steps may be performed in different orders; and (4) certain steps may be done concurrently.

Any headings used herein are for organizational purposes only and shall not be used to limit the scope of the description or the claims. Each reference/document mentioned in this patent document is incorporated by reference herein in its entirety.

It shall be noted that any experiments and results provided herein are provided by way of illustration and were performed under specific conditions using a specific embodiment or embodiments; accordingly, neither these experiments nor their results shall be used to limit the scope of the disclosure of the current patent document.

It shall also be noted that although embodiments described herein may be within the context of SARS-CoV-2 Genome, aspects of the present disclosure are not so limited. Accordingly, the aspects of the present disclosure may be applied or adapted for use in other genetic material, or non-genetic material.

### A. General Introduction

The 2019 Novel Coronavirus (SARS-CoV-2) caused an outbreak of viral pneumonia since late 2019 and has become a global pandemic. Despite efforts to contain its spread, SARS-CoV-2 has infected millions of patients and more than 800,000 deaths worldwide as of late August. To understand its evolution and genetics, scientists have sequenced SARS-CoV-2 genomes from patients across different age groups, genders, ethnicities, locations, and disease stages. These genomic sequences are being shared on public repositories at a rapid pace, with thousands of new sequences every week. To keep up with the latest developments, scientists need to frequently download and clean new datasets, which is ad hoc and time-consuming. On the other hand, scientists with limited knowledge in bioinformatics or programming may experience difficulty in analyzing SARS-CoV-2 genomes.

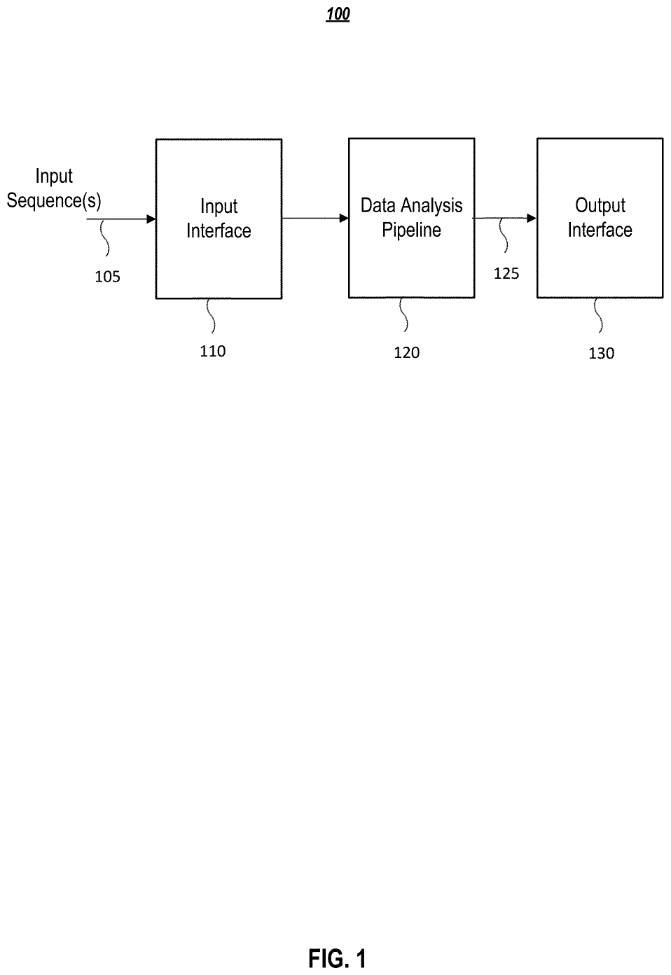

FIG. 1 depicts for a system for genome analysis and visualization, according to embodiments of the present disclosure. Specifically, one or more embodiments may be related to SARS-CoV-2 genome analysis to address aforementioned challenges and may be referred as a CoV-Seq system hereinafter. In one or more embodiments, a CoV-Seq system may comprise a data analysis pipeline 120 that takes one or more input sequences 105 via an input interface 110 and generates one or more analysis results 125, which are rendered via an output interface 130 in one or more desired formats. In one or more embodiments, the one or more desired formats may be a graphic format and/or a tabulated format. In one or more embodiments, the output interface 130 may be a web interface that a user may interact for desired rendering information.

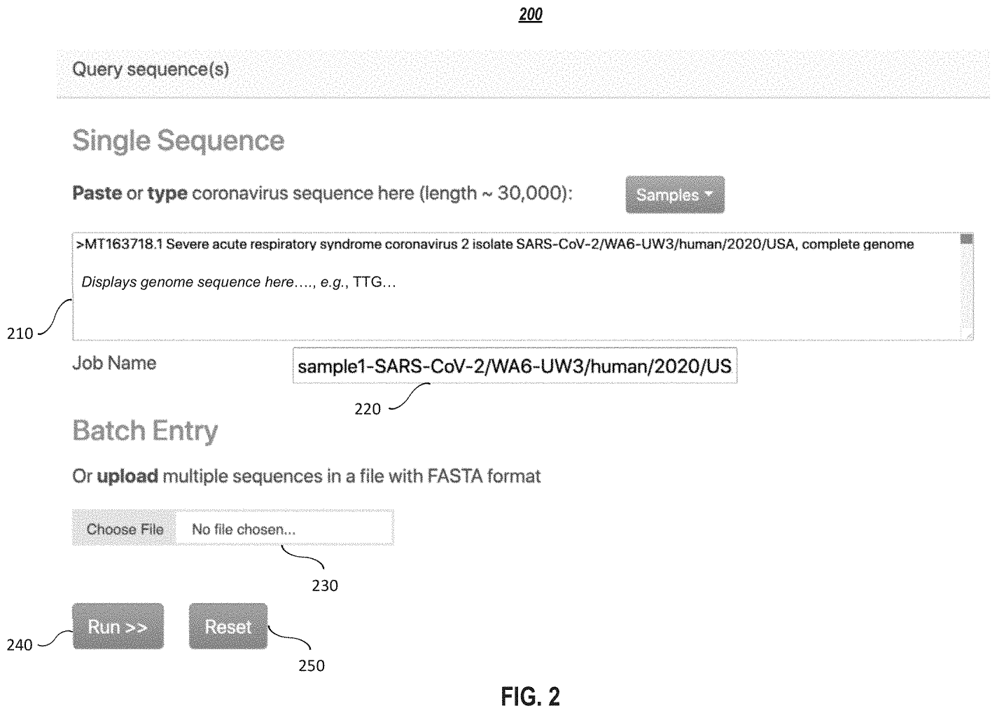

In one or more embodiments, the input interface may be a web interface accessible by a user via a web browser. FIG. 2 graphically depicts an input interface for sequence input, according to embodiments of the present disclosure. The input interface may comprise a first input box 210 for a user to input, e.g., paste or type, one or more genome sequences and a second input box 220 to receive one or more names or titles for the one or more genome sequences. A genome sequence is a chain of nucleotides, which may be expressed as symbols for representation. For example, the nucleotide may be an adenine (A), a cytosine (C), a guanine (G), a thymine (T), an uracil (U), etc. Accordingly, an example genome sequence may be expressed as “TTGGTT . . . ” Difference species may have genome sequences of different length.

Alternatively, a user may input both a sequence name and a genome sequence in one input box. In one or more embodiments, the input interface may comprise a third input box 230 to allow a user to upload one or more files with each file corresponding to one or more sequences. In one or more embodiments, it may be desirable that the one or more sequences input to the input interface have a FASTA format, a text-based format widely used in bioinformatics and biochemistry for representing nucleotide sequences and/or amino acid (protein) sequences. In one or more embodiments, the input interface may further comprise a button (e.g., a Run button) 240 for a user to initiate genome analysis for the one or more input sequences. In one or more embodiments, the input interface may further comprise a button (e.g., a Reset button) 250 to allow a user resetting the input box for another input action.

In one or more embodiments, the data analysis pipeline may automatically filter low-quality sequences and remove duplicate sequences, perform sequence alignment, as well as identify and annotate genetic variants. In one or more embodiments, a webserver may be used in the data analysis pipeline to allow rapid analysis of custom sequences without any programming. In one or more embodiments, the analysis result 125 may comprise one or more variant callsets in a desired format, e.g., a VCF, one or more ORF predictions, and/or one or more amino acid and nucleotide sequences. In one or more embodiments, a variant call may be referred as a conclusion that there is a nucleotide difference against some reference at a given position in an individual genome or transcriptome. A variant call may be accompanied by an estimate of variant frequency and some measure of confidence. A VCF is a format of a text file used in bioinformatics for storing genetic sequence variations. In one or more embodiments, a VCF file may comprise one or more meta-information lines, a VCF header line, and one or more data lines containing marker and genotype data. Each data line corresponds to one variant and may be referred as a VCF record. Each VCF record has the same number of tab-separated fields as the header line. In one or more embodiments, an ORF is a reading frame that has the potential to be transcribed into RNA and translated into protein. An ORF may require a continuous sequence of DNA from a start codon, through a subsequent region which usually has a length that is a multiple of 3 nucleotides, to a stop codon in the same reading frame.

In one or more embodiments, the output interfaces 130 may be a web interface comprising a genome visualizer and tabulated displays of genetic variants and ORF predictions. The genome visualizer may be interactive to show ORFs and mutations. In one or more embodiments, analysis results may be downloaded for downstream analysis. In one more embodiments, the input interfaces 110 and/or the output interface 130 may comprise a command-line interface to allow high-throughput processing on local environments. To facilitate data sharing, SARS-CoV-2 sequences may be aggregated from various sources, e.g., GISAID (a global science initiative and source providing open-access to genomic data of various viruses), National Center for Biotechnology Information (NCBI), European Nucleotide Archive (ENA) and China National GeneBank (CNGB), with annotated variant callsets and metadata updated periodically, e.g., on a weekly basis.

### B. Embodiments for Analysis and Visualization of Viral Genomes

The collection of SARS-CoV-2 genomic sequences is rapidly expanding. An integrated pipeline is essential for keeping current with frequent updates. To understand the evolution and genetics of a new viral strain, it may be necessary to identify its genetic mutations and gene boundaries. Some existing software packages focus on gene annotation. Solution packages to identify, annotate, and visualize genetic variants for a genome, e.g., SARS-CoV-2, may be lacking.

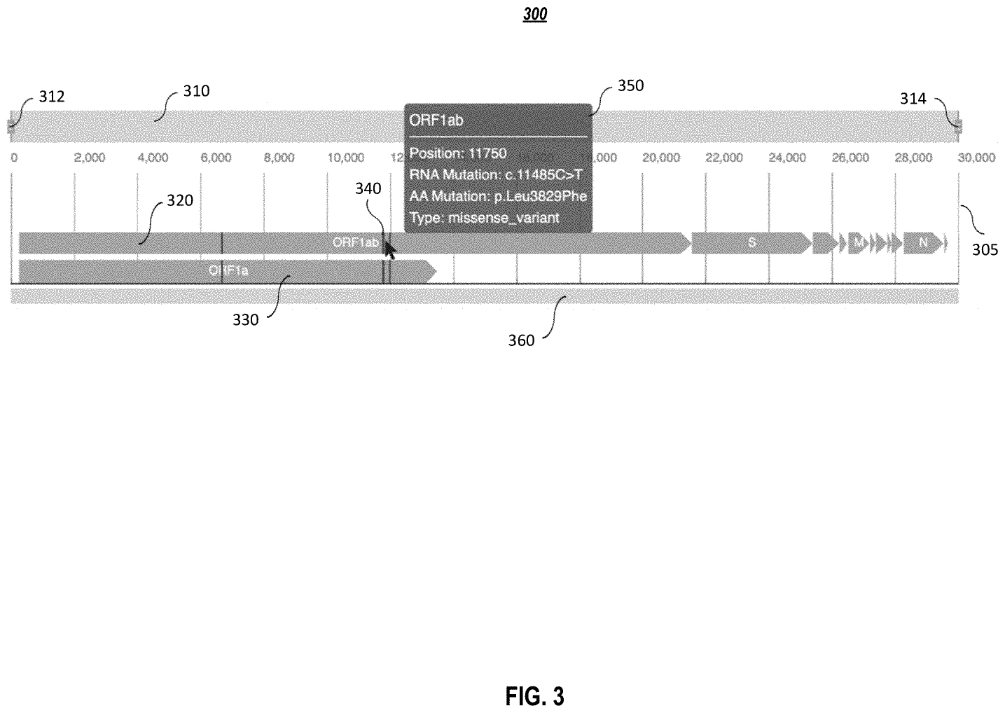

One or more embodiments of the present disclosure provide an intuitive web interface for analyzing and visualizing genome, e.g., SARS-CoV-2, variants, and ORFs. FIG. 2 depicts an interactive genome visualizer, according to embodiments of the present disclosure. Upon receiving an input sequence, the data analysis pipeline performs genome analysis to identify gene boundaries and genetic variants for the input sequences. One or more analysis results may be displayed alongside a full-length genome as shown in FIG. 3. In one or more embodiments, one or more input sequences may be collected from a database, such as GISAID, NCBI, ENA, or CNGB, or updated from a third-party, e.g., a medical center, interested in getting more information for a newly found sequence.

As shown in FIG. 3, the genome visualizer may comprise a genome window 305 and a zoom bar 310 with a starting pin 312 and an end pin 314. The genome visualizer may also comprise a position bar 360 corresponding to current position along the genome. In one or more embodiments, the zoom bar 310 may show a full-length genome for the input sequence by default. The genome length bar 310 may have numbers beneath to show numeric values of nucleotides along the bar. In one or more embodiments, a user may interact with the genome by adjusting the starting/end pins of the zoom bar to adjust the magnification and the position bar 360 to pan along the genome sequence. In one or more embodiments, the genome visualizer comprises one or more ORFs, e.g., 320 and 330, and one or more mutations, e.g., 340, displayed with marks for position identification on the genome length. Each ORF spans from a start codon to a stop codon. In one or more embodiments, when two ORFs have overlaps in sequence span, e.g., as ORF 320 and ORF 330 shown in FIG. 3, there may be arranged in different rows for visualization clarity. In one or more embodiments, the one or more ORFs, e.g., 320 and 330, shown in the genome visualizer are selected from a group of ORFs inferenced from an input sequence. In one or more embodiments, one or more ORF annotations shown in FIG. 3 may be transferred from ORF annotations for reference sequences by aligning two corresponding sequences. Certain ORF annotations from the reference sequence may not be transferred to the input sequence if insufficient similarity (e.g., less than a similarity threshold) is observed within the genomic region overlapping these ORFs.

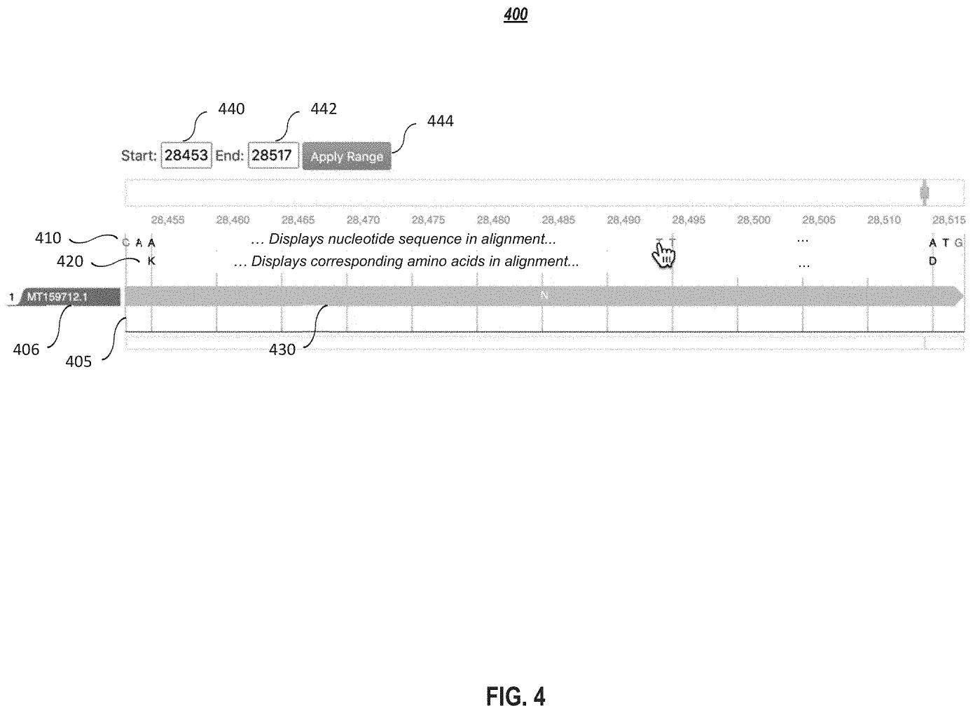

In one or more embodiments, when a cursor is hovered over ORFs and mutations, one or more pop-up windows may be triggered for relevant information. For example, the exemplary pop-up window 350 corresponding to the mutation 340 shows name of ORF (ORF lab) that the mutation belongs to, position of the mutation, RNA or AA information related to the mutation, etc. In one or more embodiments, when the genome window contains less than a predetermined number (e.g., 150) of nucleotides, nucleotide symbols may appear to indicate both the nucleotide bases and AA residues, as shown in FIG. 4

Referring to FIG. 4, a nucleotide symbol chain 410 and a corresponding AA residue chain 420 appears in a genome window 405, when there are less than a predetermined number (e.g., 150) of nucleotides in the genome window. Each AA residue corresponds to three nucleotides (a trinucleotide). For example, a lysine AA (symbolled as K in the AA residue chain 420) corresponds to a trinucleotide AAA or AAG; a glutamic acid AA (symbolled as E in the AA residue chain 420) corresponds to a trinucleotide GAA or GAG; an aspartic acid AA (symbolled as D in the AA residue chain 420) corresponds to a trinucleotide GAC or GAG, etc. In one or more embodiments, the AA residue chain is in parallel to the nucleotide symbol chain with each AA residue in line with the first, middle, the last nucleotide of the corresponding trinucleotide. For example, the AA residue of Serine (symbolled as S) may be positioned in line with nucleotide “G” in a corresponding trinucleotide AGC. In one or more embodiments, the nucleotide symbol chain 410 and a corresponding AA residue chain 420 may adopt alternative trinucleotide (and corresponding AA residue) colors for better data presentation. For example, a first trinucleotide may have a black color, while a following trinucleotide may have a grey color. In one or more embodiments, one or more ORFs 430 which comprise one or more nucleotides shown in the genome window are displayed, and one or more mutations within a nucleotide range of the genome window are also displayed. The nucleotide range of the genome window may be set by adjusting a starting pin and an end pin on a zoom bar (shown in FIG. 3), or by manually inputting a start number in a start number input box 440 and an end number input box 442 followed by an action to apply the range (e.g., by clicking a “Apply Range” button 444 next the end number input box 442). In one or more embodiments, a name tag 406 may be displayed besides the genome window to identity an association relation between one or more displayed ORFs 430 and the genome sequence designated by the name tag 406. In one or more embodiments, when multiple genome sequences are rendered in the genome window 405, a nucleotide symbol chain 410 and a corresponding AA residue chain 420 for the first (or top) genome sequence are rendered by default. A user may choose to render nucleotide symbol chains and corresponding AA residue chains for other genome sequences by clicking the tags or ORFs for those sequences or keeping a mouse cursor on the tags or ORFs for those sequences for a time longer than a predetermined period.

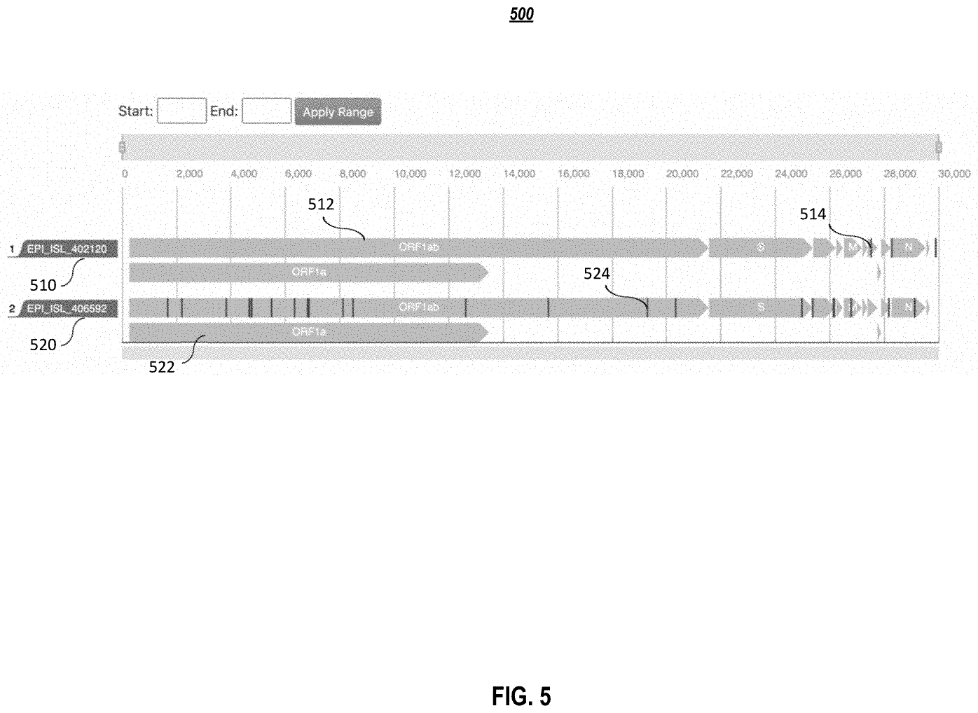

In one or more embodiments, the genome window may display ORFs associated with multiple genome sequences, e.g., sequences labeled using a first sequence tag 510 and a second sequence 520 as shown in FIG. 5. The first genome sequence may have one or more ORFs, e.g., ORF 512, and one or more mutations, e.g., mutation 514. Similarly, the second genome sequence may have one or more ORFs, e.g., ORF 522, and one or more mutations, e.g., mutation 524.

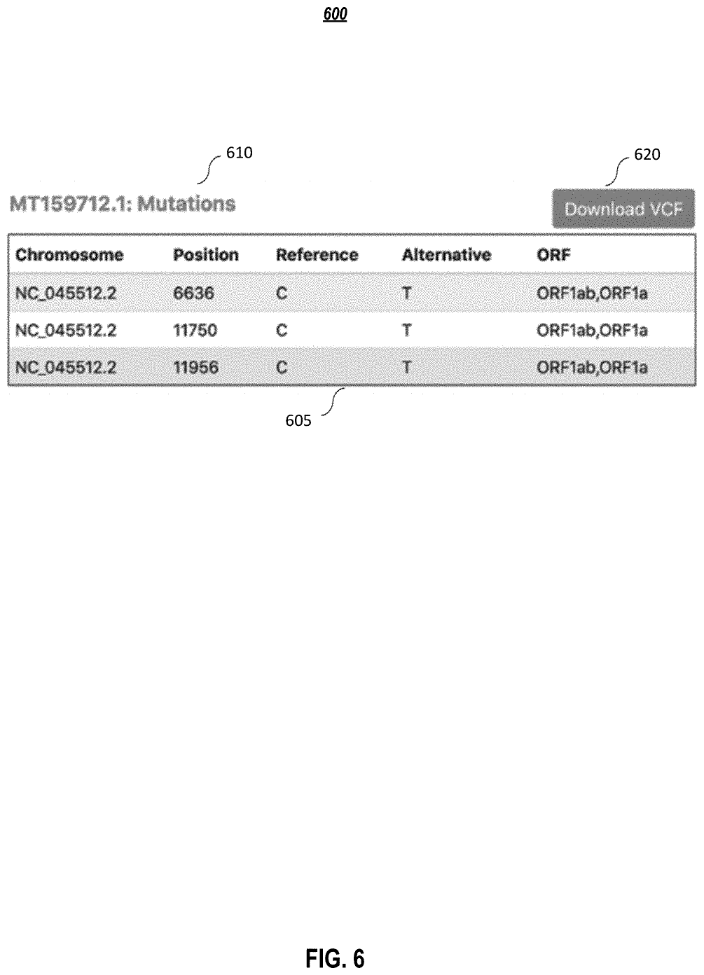

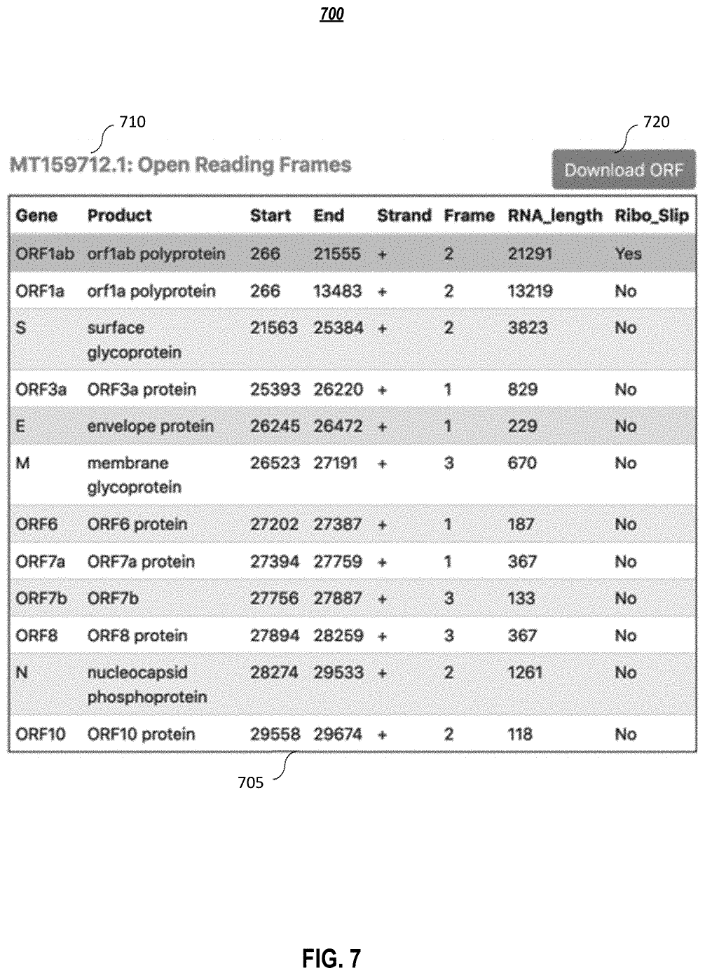

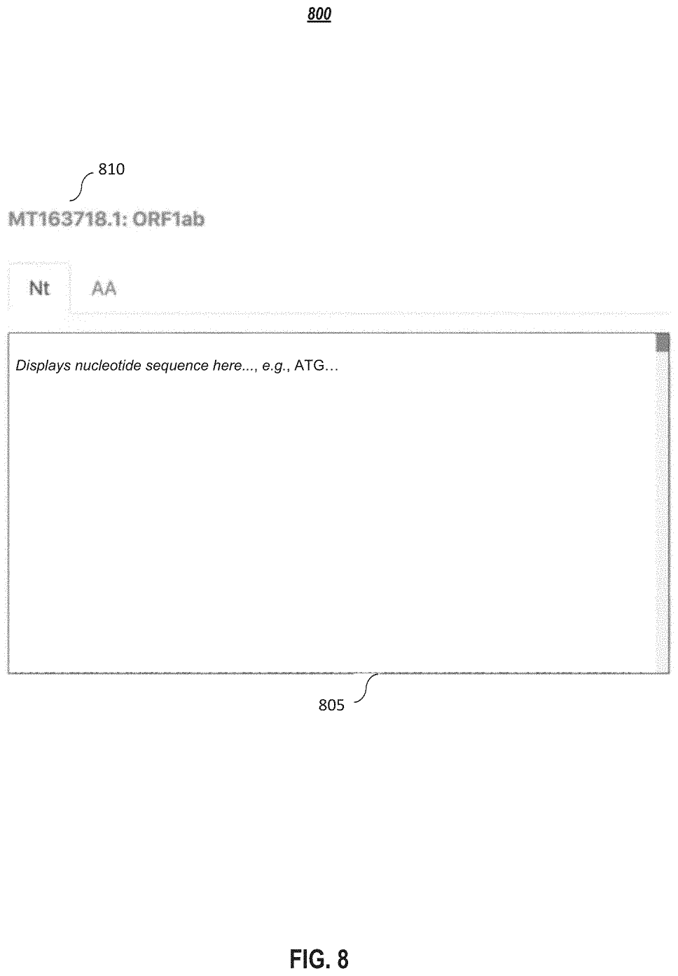

In one or more embodiments, clicking an ORF or a mutation (or keeping a cursor or a mouse pointer on an ORF or a mutation longer than a predetermined time, e.g., 2 seconds) brings up one or more tables. FIG. 6 shows a mutation table 605 comprising all variants and their positions, chromosome, alleles, and ORFs in which they belong, according to embodiments of the present disclosure. In one or more embodiments, the table 605 has a table title 610 comprising a sequence name (e.g., MT15912.1) and a table summary (e.g., mutations). FIG. 7 shows an ORFs table 705 comprising ORF annotations obtained by aligning input sequences against the reference sequence and transferring annotations from GenBank, according to embodiments of the present disclosure. In one or more embodiments, for each ORF, the table 705 lists information comprising gene name, product, start number, end number, strand, frame number, RNA length, and whether the ORF has ribosomal slippage, a ribosomal frameshifting in translating an RNA sequence. In one or more embodiments, the table 705 has a table title 710 comprising a sequence name (e.g., MT15912.1) and a table summary (e.g., open reading frames). FIG. 8 shows an individual ORF table 805 comprising nucleotide and protein sequences for the selected ORF. In one or more embodiments, the table in FIG. 8 has a table title 810 comprising a sequence name (e.g., MT163718.1) and a table summary (e.g., ORFlab).

In one or more embodiments, one or more tables may be downloadable for downstream analysis. For example, the table shown in FIG. 6 may be downloaded (e.g., via a click of a download button 620) as a VCF file, which may be further annotated with SnpEff, an open source tool that annotates variants and predicts their effects on genes by using an interval forest approach. In another example, the table shown in FIG. 7 may be downloaded (e.g., via a click of a download button 720) in a desired format, e.g., in a tab-separated values (TSV) file.

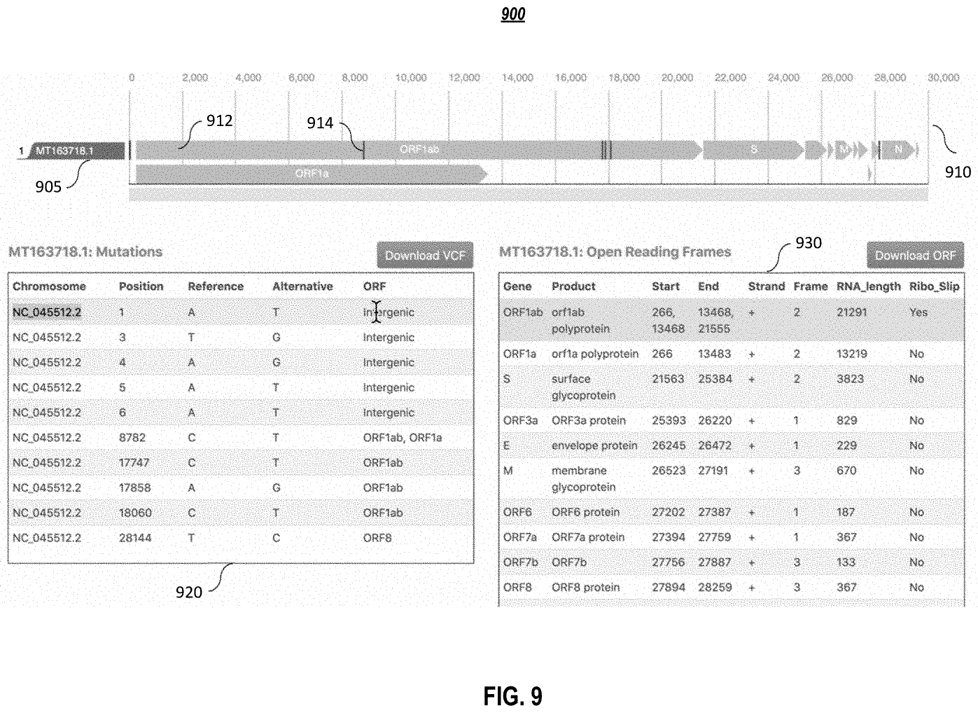

In one or more embodiments, a genome visualizer and one or more tables may be rendered together as shown in FIG. 9. When analysis results for a sequence 905 are presented, the output interface may comprise a genome visualizer window 910 and one or more tables, e.g., table 920 and table 930. The genome visualizer window 910 may display one or more ORFs 912 and mutations 914. In one or more embodiments, the one or more ORFs may be presented as a labeled strip spanning from a start codon (corresponding to a start number) to a stop codon (corresponding to an end number). In one or more embodiments, the start of an ORF may be identified by a flat end, while the end of the ORF may be identified as a sharp end, as shown in the ORF 912.

In one or more embodiments, when analysis results for the sequence 905 are initially presented, only the genome visualizer is displayed as default. When a user chooses a target ORF, one or more tables like table 920 and table 930 may be dynamically rendered for relevant information depending on which ORF the user clicks. The one or more tables dynamically shown may be pop-up tables or tables appearing on the same webpage as a replacement for the one or more default tables.

In one or more embodiments, when the genome visualizer comprises two or more sequences (as shown in FIG. 5), no tables are rendered initially or by default. When a user moves the cursor, one or more tables may be dynamically shown, as a pop-up or appearing in the same webpage, for relevant information depending on where the cursor stops or what the user clicks.

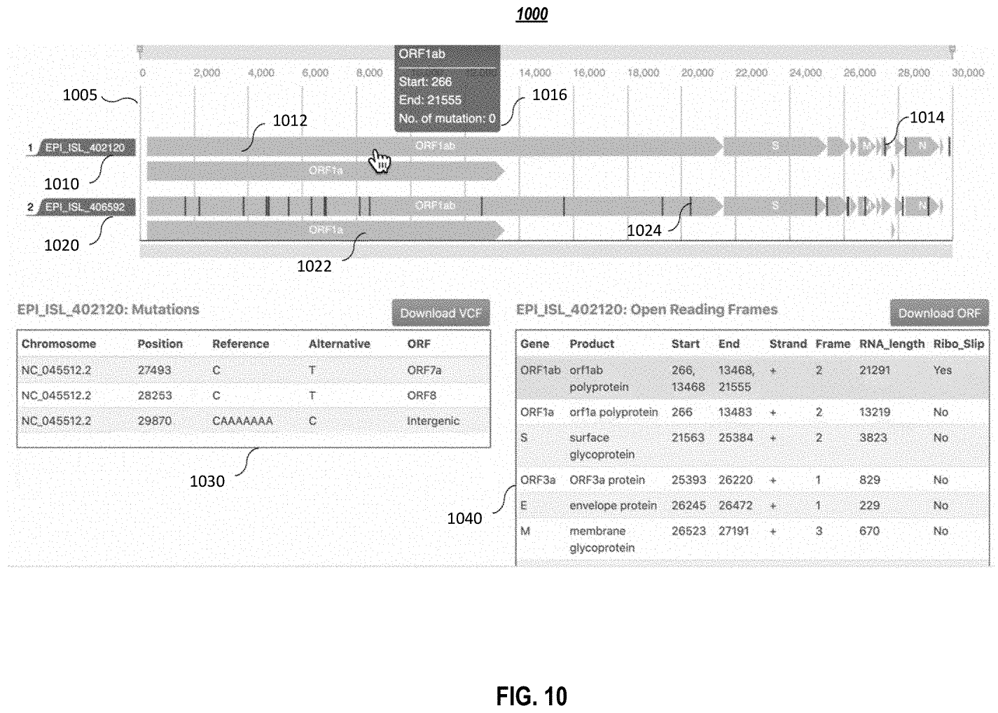

FIG. 10 depicts a genome visualizer and one or more tables dynamically rendered according to a user action, according to embodiments of the present disclosure. One or more tables may be dynamically rendered according to information related to the user interaction, which may be a click for an ORF, a click for a mutation, or a click for a tag associated to a genomic sequence. As shown in FIG. 10, the genome visualizer window 1005 shows two sequences 1010 and 1020 with each sequence associated with one or more ORFs and one or more mutations. For example, the first sequence 1010 (tagged as EPI_ISL_402120) is associated with one or more ORFs, e.g., ORF 1012, and one or more mutations, e.g., mutation 1014. The second sequence 1020 (tagged as EPI_ISL_406592) is associated with one or more ORFs, e.g., ORF 1022, and one or more mutations, e.g., mutation 1024. When a cursor stays in the ORF 1012 longer than a predetermined period, a pop-up window 1016 appears in proximity of the ORF 1016 showing information, e.g., ORF name, start number, end number and number of mutations in the ORF, etc., relevant to the ORF 1016. Furthermore, one or more tables may appear along with the genome visualizer. In one or more embodiments, the one or more tables may include a mutation table 1030 identified by a table title showing sequence to which the ORF 1012 belongs followed by a table summary (e.g., mutations). In one or more embodiments, the one or more tables may also include an ORF table 1040 identified by a table title showing sequence to which the ORF 1012 belongs followed by a table summary (e.g., open reading frames). In one or more embodiments, the one or more tables may also include a table of ORFs 1040 identified by a table title showing sequence to which the ORF 1012 belongs followed by a table summary (e.g., open reading frames). The table of ORFs may include all ORFs (not limited to the ORF 1012 that the user clicks). In one or more embodiments, the one or more tables may also include an ORF table (similar to the table shown in FIG. 8) identified by a table title showing sequence to which the ORF 1012 belongs followed by a table summary (e.g., name of the ORF that the use selects). The table of ORFs may include all ORFs (not limited to the ORF 1012 that the user clicks). The ORF table may comprise nucleotide and protein sequences for the selected ORF 1012.

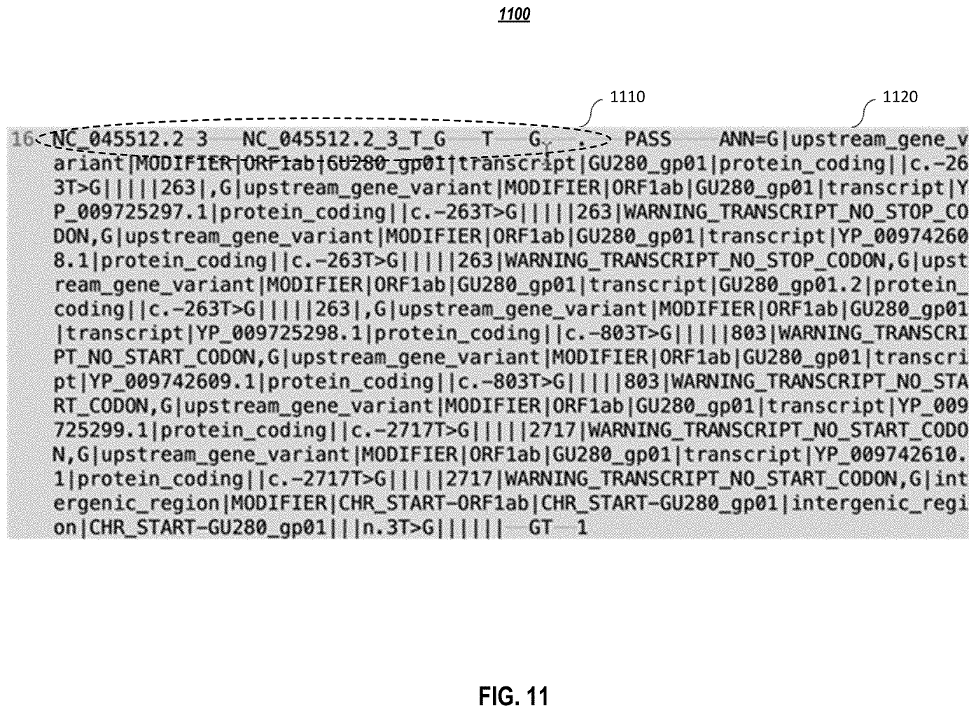

In one or more embodiments, the table 1030 may be downloaded as a VCF file, and the table 1040 may be downloaded in a desired format, e.g., in a TSV file. FIG. 11 depicts a VCF file, according to embodiments of the present disclosure. The VCF file comprise a first section 1710 corresponding information, e.g., chromosome name, position, reference allele, alternative alleles, etc., which are shown in the mutation table. The VCF file comprise a second section 1120, an annotation section which may be annotated with SnpEff, an open source tool that annotates variants and predicts their effects on genes by using an interval forest approach.

In one or more embodiments, when a user clicks a mutation, e.g., the mutation 1024, a pop-up window may appear in proximity of the mutation 1024 showing information, e.g., intersecting ORF name, RNA mutation information, AA mutation information, etc., relevant to the mutation 1024. Furthermore, one or more tables may appear along with the genome visualizer. The one or more tables may comprise a mutation table for all mutations in the sequence 1020 (since the mutation 1024 is related to the sequence 1020), a table of ORFs for all ORFs in the in the sequence 1020, and an ORF table for ORFlab of the sequence 1020 (since the mutation 1024 is related to the ORFlab in the sequence 1020).

One skilled in the art shall understand the aforementioned embodiments for genome visualization may be used in individually, in combination or sub-combination, or in combination with additional visualization approach for presentation and downstream analysis.

### C. Embodiments of Genome Processing Pipeline

In one or more embodiments, extraction of genetic variants from sequences may involve the following steps: (1) preprocessing: removal of low-quality and duplicated sequences; (2) alignment: pairwise alignment of each sequence against a reference; (3) variant calling: identify differences between aligned sequences; and (4) post-processing: remove low-quality sites and annotate variants.

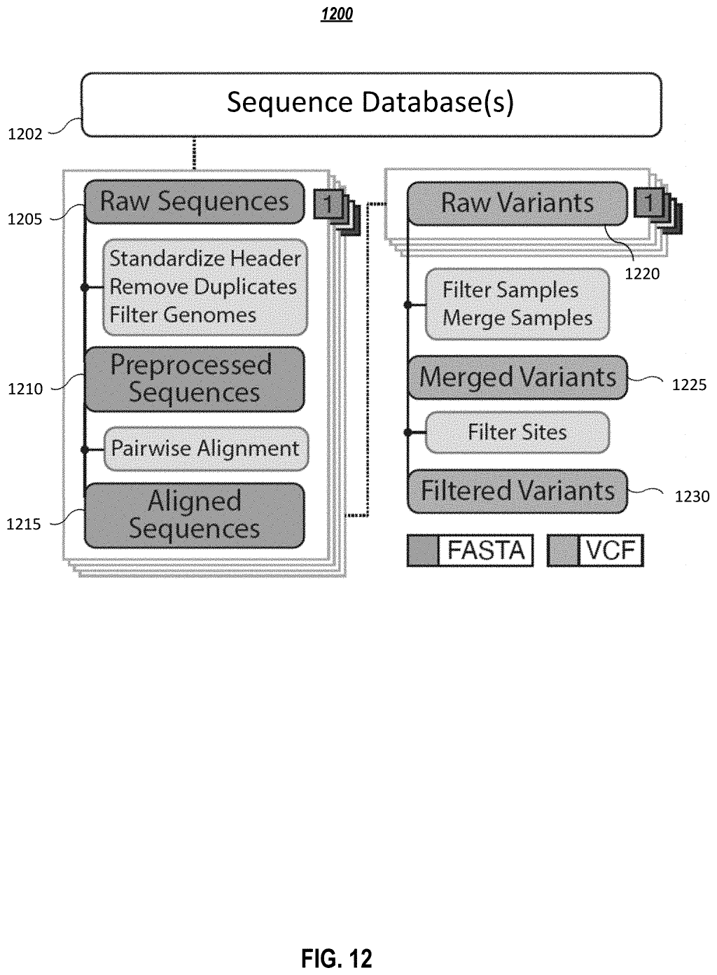

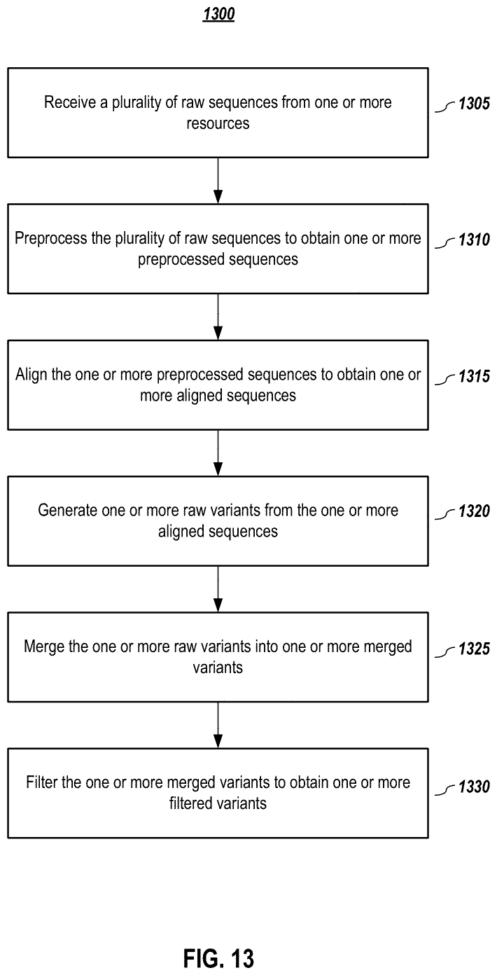

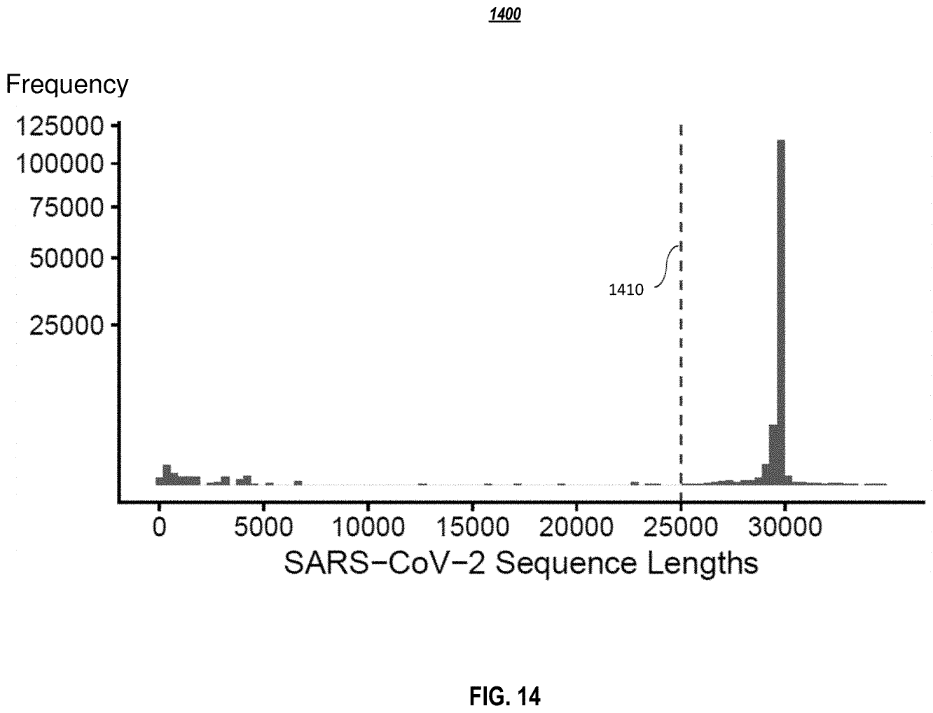

FIG. 12 graphically depicts a data analysis pipeline and FIG. 13 depicts a corresponding process of genome analysis in the data analysis pipeline, according to embodiments of the present disclosure. As shown in FIG. 12 and FIG. 13, a plurality of raw genomic sequences 1205 are received (1305) in the data analysis pipeline. The plurality of raw sequences may be aggregated from multiple resources comprising GISAID, NCBI, ENA and/or CNGB. The plurality of raw sequences may also be uploaded from a third-party, e.g., a medical center, interested in getting more information for some newly found sequences. In one or more embodiments, the plurality of raw sequences may have a FASTA format. The plurality of raw sequences are preprocessed (1310) to obtain one or more preprocessed sequences 1210. In one or more embodiments, preprocessing the one or more raw sequences may comprise standardizing header, and/or removing duplicate genomes, and/or filtering one or more incomplete genomes. In certain situations, some raw sequences may represent incomplete genomes, sometimes containing only a single gene. In one or more embodiments, incomplete genomes may be filtered using a predetermined cutoff, e.g., 25,000 nucleotides. Considering that a SARS-CoV-2 has around 30,000 nucleotides in genome length, a cutoff of 25,000 may be able to remove distinctly incomplete genomes while retains complete genomes. FIG. 14 depicts a distribution of SARS-CoV-2 sequence lengths with the dashed line 1410 representing the cutoff. Sequences with lengths less than 25,000 nucleotides are removed during preprocessing.

In one or more embodiments, one or more preprocessed sequences 1210 are aligned (1315) to obtain one or more aligned sequences 1215. In one or more embodiments, the alignment may be a pairwise alignment to identify regions of similarity that may indicate functional, structural and/or evolutionary relationships between two preprocessed sequences. One or more raw variant calls 1220 may be generated (1320), e.g., using a Python script, from the one or more one or more aligned sequences. In one or more embodiments, raw variant calls are generated in a VCF. In one or more embodiments, the Python script may be customized according to characteristics of the raw sequences. The one or more raw variant calls are merged (1325) into one or more merged variants 1225, with raw variant calls having excessive mutations (e.g., above a threshold) indicative of sequencing error removed. A filtered set of variants comprising one or more filtered variants 1230 is obtained (1330) from the one or more merged variants by filtering out merged variants associated with one or more predetermined structures. In one or more embodiments, a merged variant to be filtered out may be a multi-allelic site or a variant within the poly-A tail. In one or more embodiments, a multi-allelic site may be referred as a specific locus in a genome that contains three or more observed alleles, again counting the reference as one, and therefore allowing for two or more variant alleles. In one or more embodiments, a poly-A tail may be referred as a chain of adenine nucleotides that is added to a messenger RNA (mRNA) molecule during RNA processing to increase the stability of the molecule. In one or more embodiments, the one or more filtered variants are the analysis results from the data analysis pipeline.

In one or more embodiments, all the sequences, e.g., the raw sequences, preprocessed sequences, and the aligned sequences, shown in FIG. 12 are in a FASTA format, while the variants shown in FIG. 12, e.g., the raw variants, merged variant, and the filtered variants, are in a VCF format.

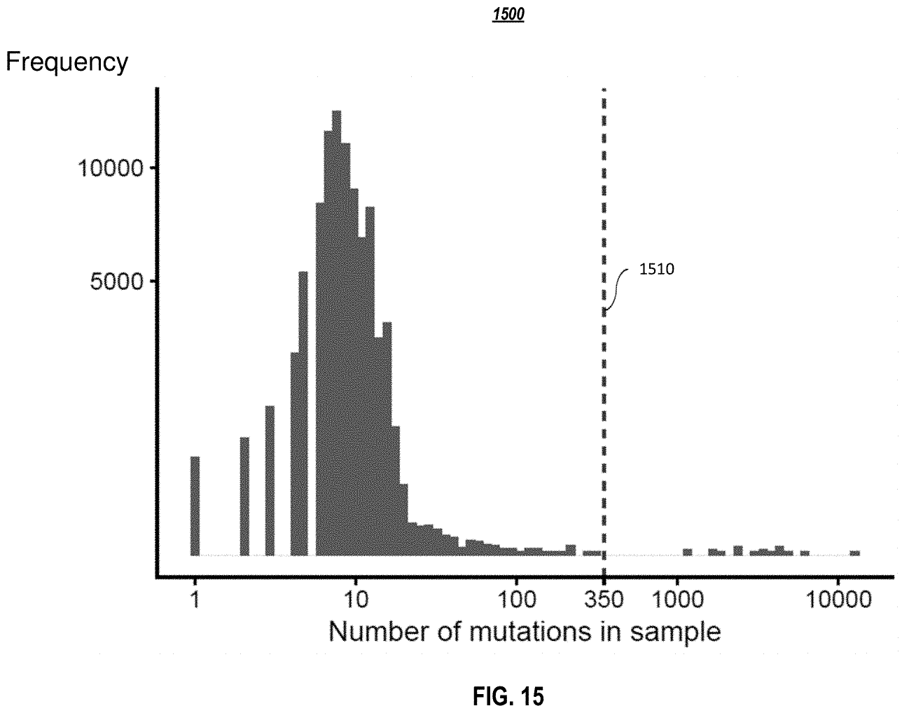

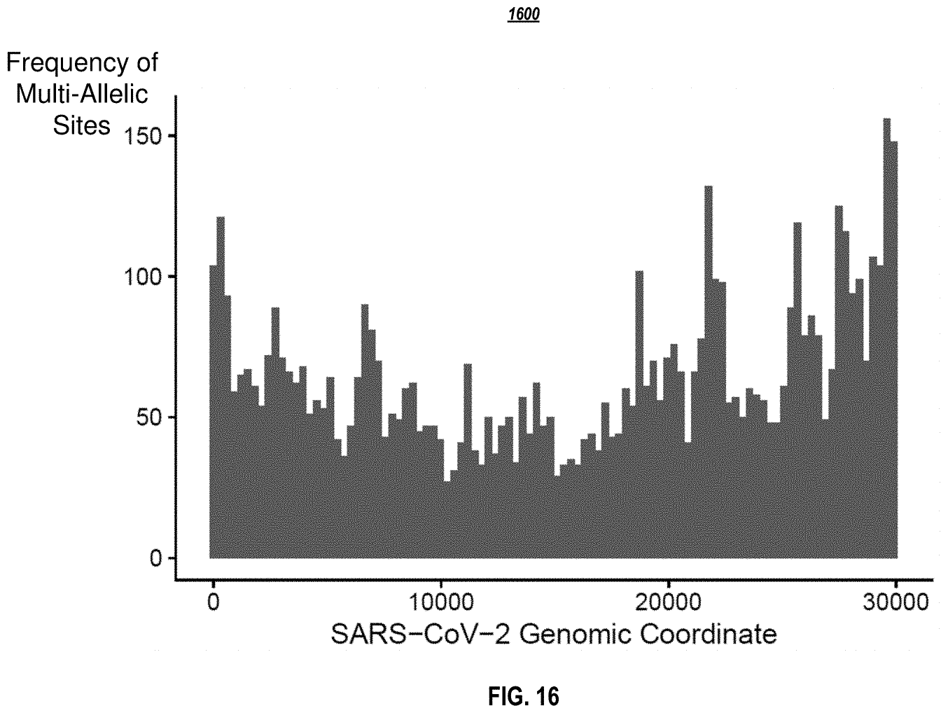

Regarding the resources for raw sequences, both NCBI and ENA are part of the International Nucleotide Sequence Database Collaboration (INSDC) and therefore may contain duplicate submissions, which may be removed by comparing the Accession IDs in one or more embodiments of the present disclosure. Further, dual submissions may also appear in both GISAID and INSDC under different Accession IDs. In one or more embodiments, two submissions may be considered as suspect duplications if they have identical genomic sequences. These suspect duplications may be marked in the metadata but not removed because it is possible for an identical strain to infect multiple patients. In one or more embodiments, pairwise alignment may be performed against a reference sequence, e.g., NC_045512.2, using MAFFT (for multiple alignment using fast Fourier transform), a program used to create multiple sequence alignments of amino acid or nucleotide sequences. In one or more embodiments, a custom Python script may be used for variant calling. Each variant may be left-normalized with bcftools, a set of utilities that manipulate variant calls in the VCF and its binary counterpart BCF. samples with too many variants indicative of sequencing error may be removed. In one or more embodiments, a lenient cutoff of 150 variants is used because such a cut off may remove samples with extremely large numbers of variants while keeping most samples. FIG. 15 depicts a distribution of sample mutations identified against a reference genome NC_045512.2, according to embodiments of the present disclosure. Samples with more than 350 mutations (dashed line 1510) removed during post-processing. In one or more embodiments, multi-allelic sites are removed because these sites are more likely to occur in regions prone to sequencing error, such as the two ends of the genome. FIG. 16 depicts a distribution of multi-allelic sites along the SARS-CoV-2 genome, according to embodiments of the present disclosure. As shown in FIG. 16, multi-allelic sites are more likely to be identified at the beginning and the end of the genome. Further, variants within the poly-A tail may also be removed. Filtered variant callset may be annotated with SnpEff. In one or more embodiments, both raw and filtered VCF files may be downloadable and the pipeline may be open source. The VCF files and associated metadata may be updated periodically, e.g., weekly, to keep in pace of genomic sequences sharing.

In one or more embodiments, the data analysis pipeline may be hosted in a server or a cloud accessible to a user via network communication. In one or more embodiments, the data analysis pipeline may be hosted in a local environment, which may be run without network connection. In one or more embodiments, a standalone package for genome analysis and visualization may be downloadable, e.g., from a GitHub repository, for a user to download and run locally.

### D. Computing System Embodiments

In one or more embodiments, aspects of the present patent document may be directed to, may include, or may be implemented on one or more information handling systems (or computing systems). An information handling system/computing system may include any instrumentality or aggregate of instrumentalities operable to compute, calculate, determine, classify, process, transmit, receive, retrieve, originate, route, switch, store, display, communicate, manifest, detect, record, reproduce, handle, or utilize any form of information, intelligence, or data. For example, a computing system may be or may include a personal computer (e.g., laptop), tablet computer, mobile device (e.g., personal digital assistant (PDA), smart phone, phablet, tablet, etc.), smart watch, server (e.g., blade server or rack server), a network storage device, camera, or any other suitable device and may vary in size, shape, performance, functionality, and price. The computing system may include random access memory (RAM), one or more processing resources such as a central processing unit (CPU) or hardware or software control logic, read only memory (ROM), and/or other types of memory. Additional components of the computing system may include one or more disk drives, one or more network ports for communicating with external devices as well as various input and output (I/O) devices, such as a keyboard, mouse, stylus, touchscreen and/or video display. The computing system may also include one or more buses operable to transmit communications between the various hardware components.

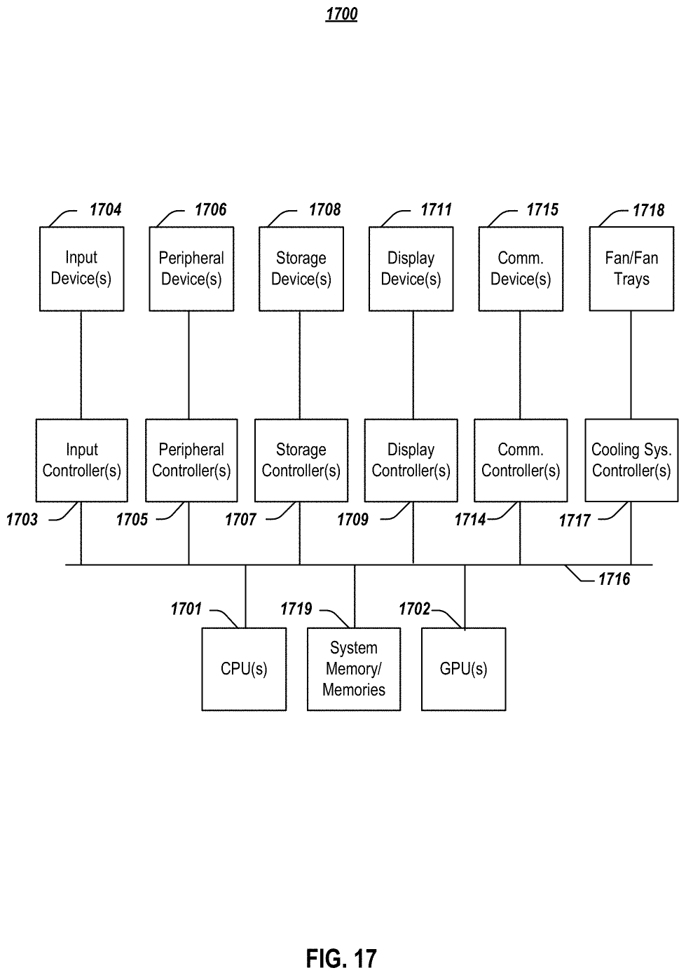

FIG. 17 depicts a simplified block diagram of an information handling system (or computing system), according to embodiments of the present disclosure. It will be understood that the functionalities shown for system 1700 may operate to support various embodiments of a computing system—although it shall be understood that a computing system may be differently configured and include different components, including having fewer or more components as depicted in FIG. 17.

As illustrated in FIG. 17, the computing system 1700 includes one or more central processing units (CPU) 1701 that provides computing resources and controls the computer. CPU 1701 may be implemented with a microprocessor or the like, and may also include one or more graphics processing units (GPU) 1702 and/or a floating-point coprocessor for mathematical computations. In one or more embodiments, one or more GPUs 1702 may be incorporated within the display controller 1709, such as part of a graphics card or cards. Thy system 1700 may also include a system memory 1719, which may comprise RAM, ROM, or both.

A number of controllers and peripheral devices may also be provided, as shown in FIG. 17. An input controller 1703 represents an interface to various input device(s) 1704, such as a keyboard, mouse, touchscreen, and/or stylus. The computing system 1700 may also include a storage controller 1707 for interfacing with one or more storage devices 1708 each of which includes a storage medium such as magnetic tape or disk, or an optical medium that might be used to record programs of instructions for operating systems, utilities, and applications, which may include embodiments of programs that implement various aspects of the present disclosure. Storage device(s) 1708 may also be used to store processed data or data to be processed in accordance with the disclosure. The system 1700 may also include a display controller 1709 for providing an interface to a display device 1711, which may be a cathode ray tube (CRT) display, a thin film transistor (TFT) display, organic light-emitting diode, electroluminescent panel, plasma panel, or any other type of display. The computing system 1700 may also include one or more peripheral controllers or interfaces 1705 for one or more peripherals 1706. Examples of peripherals may include one or more printers, scanners, input devices, output devices, sensors, and the like. A communications controller 1714 may interface with one or more communication devices 1715, which enables the system 1700 to connect to remote devices through any of a variety of networks including the Internet, a cloud resource (e.g., an Ethernet cloud, a Fiber Channel over Ethernet (FCoE)/Data Center Bridging (DCB) cloud, etc.), a local area network (LAN), a wide area network (WAN), a storage area network (SAN) or through any suitable electromagnetic carrier signals including infrared signals. As shown in the depicted embodiment, the computing system 1700 comprises one or more fans or fan trays 1718 and a cooling subsystem controller or controllers 1717 that monitors thermal temperature(s) of the system 1700 (or components thereof) and operates the fans/fan trays 1718 to help regulate the temperature.

In the illustrated system, all major system components may connect to a bus 1716, which may represent more than one physical bus. However, various system components may or may not be in physical proximity to one another. For example, input data and/or output data may be remotely transmitted from one physical location to another. In addition, programs that implement various aspects of the disclosure may be accessed from a remote location (e.g., a server) over a network. Such data and/or programs may be conveyed through any of a variety of machine-readable medium including, for example: magnetic media such as hard disks, floppy disks, and magnetic tape; optical media such as CD-ROMs and holographic devices; magneto-optical media; and hardware devices that are specially configured to store or to store and execute program code, such as application specific integrated circuits (ASICs), programmable logic devices (PLDs), flash memory devices, other non-volatile memory (NVM) devices (such as 3D XPoint-based devices), and ROM and RAM devices.

Aspects of the present disclosure may be encoded upon one or more non-transitory computer-readable media with instructions for one or more processors or processing units to cause steps to be performed. It shall be noted that the one or more non-transitory computer-readable media shall include volatile and/or non-volatile memory. It shall be noted that alternative implementations are possible, including a hardware implementation or a software/hardware implementation. Hardware-implemented functions may be realized using ASIC(s), programmable arrays, digital signal processing circuitry, or the like. Accordingly, the “means” terms in any claims are intended to cover both software and hardware implementations. Similarly, the term “computer-readable medium or media” as used herein includes software and/or hardware having a program of instructions embodied thereon, or a combination thereof. With these implementation alternatives in mind, it is to be understood that the figures and accompanying description provide the functional information one skilled in the art would require to write program code (i.e., software) and/or to fabricate circuits (i.e., hardware) to perform the processing required.

It shall be noted that embodiments of the present disclosure may further relate to computer products with a non-transitory, tangible computer-readable medium that have computer code thereon for performing various computer-implemented operations. The media and computer code may be those specially designed and constructed for the purposes of the present disclosure, or they may be of the kind known or available to those having skill in the relevant arts. Examples of tangible computer-readable media include, for example: magnetic media such as hard disks, floppy disks, and magnetic tape; optical media such as CD-ROMs and holographic devices; magneto-optical media; and hardware devices that are specially configured to store or to store and execute program code, such as application specific integrated circuits (ASICs), programmable logic devices (PLDs), flash memory devices, other non-volatile memory (NVM) devices (such as 3D XPoint-based devices), and ROM and RAM devices. Examples of computer code include machine code, such as produced by a compiler, and files containing higher level code that are executed by a computer using an interpreter. Embodiments of the present disclosure may be implemented in whole or in part as machine-executable instructions that may be in program modules that are executed by a processing device. Examples of program modules include libraries, programs, routines, objects, components, and data structures. In distributed computing environments, program modules may be physically located in settings that are local, remote, or both.

One skilled in the art will recognize no computing system or programming language is critical to the practice of the present disclosure. One skilled in the art will also recognize that a number of the elements described above may be physically and/or functionally separated into modules and/or sub-modules or combined together.

It will be appreciated to those skilled in the art that the preceding examples and embodiments are exemplary and not limiting to the scope of the present disclosure. It is intended that all permutations, enhancements, equivalents, combinations, and improvements thereto that are apparent to those skilled in the art upon a reading of the specification and a study of the drawings are included within the true spirit and scope of the present disclosure. It shall also be noted that elements of any claims may be arranged differently including having multiple dependencies, configurations, and combinations.

What is claimed is:

1. A computer-implemented method for genome analysis and visualization comprising:
receiving one or more genomic sequences;
performing, using a data analysis pipeline, genome analysis to obtain one or more analysis results for each of the one or more genomic sequences, the one or more analysis results for each genomic sequence comprise one or more open reading frames (ORFs) and one or more mutations, each ORF has a boundary defined by a start codon and a stop codon, each mutation corresponds to one or more intersecting ORFs among the one or more ORFs; and
rendering, via an output interface, the one or more analysis results for each of the one or more genomic sequences in a genome visualizer, the one or more ORFs are presented with boundaries in a genome window in the genome visualizer, the one or more mutations are marked with position identification on respective one or more intersecting ORFs.

2. The computer-implemented method of claim 1 wherein the genome window has a genome length that is interactively adjustable.

3. The computer-implemented method of claim 1 further comprising:
rendering one or more tables based on a user interaction on the genome visualizer, the one or more tables are dynamically rendered according to information related to the user interaction, the user interaction is a click for an ORF, a click for a mutation, a click for a tag associated to a genomic sequence among the one or more genomic sequences, or keeping a cursor or a mouse pointer on an ORF or a mutation longer than a predetermined time.

4. The computer-implemented method of claim 3 wherein the one or more tables comprise a mutation table comprising a position, alleles, and one or more intersecting ORFs for each mutation corresponding to a genomic sequence related to the user interaction, the mutation table is downloadable as a variant callset in a variant call format (VCF).

5. The computer-implemented method of claim 3 wherein the one or more tables comprise a table of ORFs comprising information and annotations for each ORF corresponding to a genomic sequence related to the user interaction, the table of ORFs is downloadable in a tab-separated values (TSV) file.

6. The computer-implemented method of claim 3 wherein the one or more tables comprise an ORF table showing nucleotide and protein sequences corresponding to an ORF related to the user interaction.

7. The computer-implemented method of claim 2 wherein responsive to the genome length of the genome window less than a threshold, rendering a nucleotide symbol chain and a corresponding amino acid (AA) residue chain in the genome window, the nucleotide symbol chain and the corresponding AA residue chain are related to one of the one or more genomic sequences.

8. A computer-implemented method for genome analysis and visualization comprising:
receiving a plurality of genomic sequences;
preprocessing the plurality of genomic sequences to obtain one or more preprocessed sequences;
aligning the one or more preprocessed sequences to obtain one or more aligned sequences;
generating one or more raw variants from the one or more aligned sequences;
merging the one or more raw variants into one or more merged variants;
filtering the one or more merged variants to obtain one or more filtered variants; and
rendering, via an output interface, the one or more filtered variants with corresponding one or more mutations in a genome visualizer, the one or more filtered variants are graphically shown in a genome window of the genome visualizer, the one or more mutations are marked with position identification on respective one or more intersecting filtered variants.

9. The computer-implemented method of claim 7 wherein the plurality of genomic sequences are SARS-CoV-2 sequences in a FASTA format and aggregated from multiple sources sequences.

10. The computer-implemented method of claim 8 wherein preprocessing the plurality of genomic sequences comprises one or more of:
standardizing header for the plurality of genomic sequences;
removing duplicate genomes among the plurality of genomic sequences; and
filtering one or more incomplete genomic sequences.

11. The computer-implemented method of claim 10 wherein the one or more incomplete genomic sequences are genomic sequences with nucleotide length less than a cutoff.

12. The computer-implemented method of claim 8 wherein aligning the one or more preprocessed sequences comprises pairwise alignment to identify regions of similarity indicating functional, structural or evolutionary relationships between two preprocessed sequences.

13. The computer-implemented method of claim 8 wherein merging the one or more raw variants comprises removing one or more raw variants having mutations above a threshold.

14. The computer-implemented method of claim 8 wherein filtering the one or more merged variants comprising removing one or more merged variants identified as multi-allelic sites or having with a poly-A tail.

15. The computer-implemented method of claim 8 wherein the one or more filtered variants are rendered as open reading frames (ORFs) in the genome visualizer.

16. The computer-implemented method of claim 8 wherein the one or more filtered variants have a variant call format (VCF).

17. A non-transitory computer-readable medium or media comprising one or more sequences of instructions which, when executed by at least one processor, causes steps for genome analysis and visualization comprising:
receiving a plurality of genomic sequences;
preprocessing the plurality of genomic sequences to obtain one or more preprocessed sequences;
aligning the one or more preprocessed sequences to obtain one or more aligned sequences;
generating one or more raw variants from the one or more aligned sequences;
merging the one or more raw variants into one or more merged variants;
filtering the one or more merged variants to obtain one or more filtered variants; and
rendering, via an output interface, the one or more filtered variants with corresponding one or more mutations in a genome visualizer, each of the one or more filtered variants is graphically shown in a genome visualizer with a boundary along a genome length, each of the one or more mutations is marked for position identification on one or more intersecting filtered variants.

18. The non-transitory computer-readable medium or media of claim 17 wherein preprocessing the plurality of genomic sequences comprises one or more of:
standardizing header for the plurality of genomic sequences;
removing duplicate genomes among the plurality of genomic sequences; and
filtering one or more genomic sequences with nucleotide length less than a cutoff.

19. The non-transitory computer-readable medium or media of claim 17 wherein merging the one or more raw variants comprises removing one or more raw variants having mutations above a threshold.

20. The non-transitory computer-readable medium or media of claim 17 wherein filtering the one or more merged variants comprising removing one or more merged variants identified as multi-allelic sites or having with a poly-A tail.
